(19) United States
(12) Patent Application Publication
(10) Pub. No.: US 2015/0118334 A1
(43) Pub. Date: Apr. 30, 2015

(54) TOPICAL COMPOSITIONS FOR THE TREATMENT OF CHRONIC INFLAMMATORY SKIN DISEASE

(71) Applicant: KAMEDIS LTD, Tel-Aviv (IL)

(72) Inventor: Sharon Rozenblat, Tel Aviv (IL)

(21) Appl. No.: 14/408,294

(22) PCT Filed: Jun. 17, 2013
(86) PCT No.: PCT/IL2013/050516

§ 371 Date: Dec. 16, 2014

Related U.S. Application Data
(60) Provisional application No. 61/660,741, filed on Jun. 17, 2012.

(51) Int. Cl. A61K 36/708; A61K 36/539; A61K 36/234

(52) U.S. Cl. CPC A61K 36/708; A61K 36/234; A61K 36/539
USPC 424/745

(57) ABSTRACT

A topical composition comprising Rheum palmatum Root Extract, Cnidium monnieri Fruit Extract, Scutellaria baicalensis Root Extract, dipotassium glycyrrhizate, hyaluronic acid and a dermatologically acceptable carrier and use thereof for treating and/or preventing a chronic inflammatory skin disease or disorder or allergic skin conditions.

### BACKGROUND OF THE INVENTION

Atopic dermatitis (AD) is a common chronic inflammatory skin disease characterized by eczematous lesions, xerosis (dry skin) and pruritus (sensation of itching). AD usually starts in early infancy. The lifetime prevalence of AD is 10-30% in children and 1-3% in adults. The condition develops due to impaired skin barrier and altered immune reactivity. Staphylococcus aureus colonization, which occurs in 85-90% of patients, may aggravate AD due to secondary infections. Exotoxins and other substances released by S. aureus may act as allergens or superantigens. The yeasts Malassezia and Candida may also aggravate AD by triggering allergic reactions.

The mechanisms that promote the enhanced susceptibility to cutaneous infections in atopic dermatitis include skin barrier dysfunction, reduced skin lipid content, and abnormalities of the innate immune response.

As a defensive barrier, the epidermis protects internal organs from physical and chemical trauma, microorganism invasion, and ultraviolet radiation. It also aids in the regulation of transepidermal movement of water and electrolytes, including in preventing dehydration, which is essential for sustaining life. The main role of the epidermis is considered to be in the stratum corneum and the lipid matrix, located in the intercellular space. The occurrence of dysfunction in the epidermal barrier is an important factor in the physiopathogenesis of skin diseases, particularly atopic dermatitis.

The damaged barrier in AD is due to lipid depletion. The permeability barrier defect in AD is characterized by a global reduction in the contents of all three key lipids (i.e., cholesterol, FFA, and Cer), with a further reduction in ceramide content. Thus, correction of the barrier abnormality in AD requires topical applications, not only of sufficient quantities of all three key lipids that mediate barrier function, but also of lipids in a ceramide-dominant proportion that corrects the underlying lipid biochemical abnormality in AD.

Herbs that comprise anti-inflammatory and anti-allergic properties have been previously described in the art. These include Sanguisorba officinalis known in Traditional Chinese Medicine to treat eczema and pain in the skin and Silybum marianum previously described to comprise anti-inflammatory properties.

Current treatment of AD does not cure the disease but can control the severity and duration of symptoms. Such treatments include topical application of emollients and moisturizers and/or herbs to preserve and restore the compromised barrier function, cortico-steroids and calcineurin inhibitors. The latter two types of treatment are often used in acute conditions, but are not recommended for long-term use.

Therefore, there is a need for a topical composition comprising herbs for the long term treatment of atopic dermatitis.

### BRIEF DESCRIPTION OF THE DRAWINGS

FIG. 1 is a bar graph depicting the intensity change of individual symptoms of atopic dermatitis after three weeks of treatment with an exemplary composition of the invention.

FIG. 2 is a bar graph showing the percentage of patients showing improvement in the symptoms of atopic dermatitis after three weeks of treatment with an exemplary composition of the invention.

FIG. 3 is a bar graph showing the change in the average itching level of individuals with atopic dermatitis after three weeks of treatment with an exemplary composition of the invention.

FIG. 4 is a bar graph showing the Trans Epidermal Water Loss (TWEL or evaporation) (a) and skin hydration (b) after three weeks of treatment with an exemplary composition of the invention.

FIGS. 5a and 5c show the distribution of the women responses to the different parameters of the questionnaire following the application of the TOPIC Medis Face Cream of the previous formulation.

FIGS. 5b and 5d show the distribution of the women responses to the different parameters of the questionnaire following the application of the TOPIC Medis Face Cream of the new formula.

FIGS. 6a and 6c show the distribution of the women and man responses to the different parameters of the questionnaire following the application of the TOPIC Medis Body Lotion of the previous formulation.

FIGS. 6b and 6d show the distribution of the women and man responses to the different parameters of the questionnaire following the application of the TOPIC Medis Body Lotion of the new formula.

FIGS. 7a and 7c show the distribution of the women responses to the different parameters of the questionnaire following the application of the TOPIC Medis Calming Lotion of the previous formulation.

FIGS. 7b and 7d show the distribution of the women responses to the different parameters of the questionnaire following the application of the TOPIC Medis Calming Lotion of the new formula.

FIG. 8 is a bar graph showing the beneficial effect of the TOPIC Medis Extra Moisturizing Gel in treating the inflammation, redness and dryness in atopic dermatitis patients.

### SUMMARY OF THE INVENTION

In an embodiment of the invention, there is provided a topical composition comprising Rheum palmatum Root Extract, Cnidium monnieri Fruit Extract, Scutellaria baicalensis Root Extract, dipotassium glycyrrhizate, hyaluronic acid and a dermatologically acceptable carrier. In some embodiments of the invention, the concentration of the hyaluronic acid ranges from 0.001 to 0.2% w/w. In some embodiments, the concentration of the hyaluronic acid ranges from 0.01 to 0.03% w/w. The composition may further comprise cholesterol, ceramide mixture or phytosphingosine or any combination thereof. The composition may further comprise tocopheryl acetate, collodial oatmeal, glycerine, shea butter (Butyrospermum Parkii) or any combination thereof. In some embodiments of the invention, the composition may further comprise hydroxyphenyl propamidobenzoic acid, bisabolol or both. In some embodiments of the invention, the composition comprises a skin conditioning agent which may be one or more of petrolatum, caprylic/capric triglyceride, cetearyl ethylhexanoate, dimethicone, butyrospermum parkii (shea butter), mineral oil, persea gratissima (avocado) oil, phenyl trimethicone or any combination thereof. In some embodiments of the invention, the composition comprises an emulsifier and/or a viscosity increasing agent. The emulsifier and/or a viscosity increasing agent is typically selected from the group consisting of cetearyl alcohol, cetyl alcohol, cetearyl glucoside, glyceryl stearate, PEG-100 stearate, ceteareth-33, sodium acrylate/sodium acryloyldimethyl taurate copolymer, polysorbate 80, polyacrylate 13 & polyisobutene & polysorbate 20, acrylates/C10-30 alkyl acrylate crosspolymer and any combination thereof. The composition may further comprise one or more of sodium lauroyl lactylate, carbomer, xanthan gum or any combination thereof. The composition is typically in a form of a body lotion or body cream, a face cream, a calming lotion or a moisturizing gel.

According to some embodiments of the invention, there is provided a method of treating and/or preventing a chronic inflammatory skin disease or disorder or allergic skin conditions comprising the step of contacting the skin of a subject in need with the composition of the invention. Further, the invention provides the use of the composition described herein in the manufacturing of a medicament for treating and/or preventing a chronic inflammatory skin disease or disorder or allergic skin conditions.

In some embodiments, there is provided a method of treating and/or preventing a secondary infection resulted from dermatitis comprising the step of contacting the skin of a subject in need with the composition the invention.

### DESCRIPTION OF THE DETAILED EMBODIMENTS OF THE INVENTION

This invention is based on the finding that a topical composition comprising a combination of extracts from medicinal plants together with dipotassium glycyrrhizate and hyaluronic acid, was found effective in the treatment of chronic inflammatory skin diseases or disorders, such as dermatitis, especially contact dermatitis and atopic dermatitis.

The topical composition of the present invention is effective in the fields of medicaments and cosmetics.

In an embodiment of the invention, there is provided a topical composition comprising Rheum palmatum Root Extract, Cnidium monnieri Fruit Extract, Scutellaria baicalensis Root Extract, dipotassium glycyrrhizate and hyaluronic acid and a dermatologically acceptable carrier. The topical composition may be in a form of a body lotion or a cream, face cream, a gel or a calming lotion.

In some embodiments of the invention, the composition further comprises cholesterol, ceramide, or phytosphingosine or any combination thereof.

In some embodiments of the invention, the composition further comprises emollients.

The term “emollient” refers to a material useful for the prevention or relief of dryness, as well as for the protection of the skin. Many emollients are known and may be used in the composition of the invention. Examples may be found in Sagarin, Cosmetics, Science and Technology, 2nd Edition, Vol. 1, pp. 3243 (1972), which contains numerous examples of materials suitable as an emollient and is fully incorporated herein by reference. An exemplary emollient is glycerin. Additional emollients which may be used include hydrocarbon oils and waxes, such as mineral oil, petrolatum, vegetable and animal oils and fats, such as olive oil, palm oil, castor oil, corn oil, soybean oil, and lanolin and its derivatives, such as lanolin, lanolin oil, lanolin wax, lanolin alcohols, and the like. Other emollients include esters of fatty acids having 10 to 20 carbon atoms, such as including myristic, stearic, isostearic, palmitic, and the like, such as methyl myristate, propyl myristate, butyl myristate, propyl stearate, propyl isostearate, propyl palmitate, and the like. Other emollients include fatty acids having 10 to 20 carbon atoms, including stearic, myristic, lauric, isostearic, palmitic, and the like. Emollients also include fatty alcohols having ten to twenty carbon atoms, such as cetyl, myristyl, lauryl, isostearyl, stearyl and others.

According to some embodiments of the invention, the composition further comprises one or more of sodium lauroyl lactylate, carbomer, xanthan gum or any combination thereof.

According to some embodiments of the invention, the concentration of the hyaluronic acid ranges from 0.001 to 0.2% w/w.

According to some embodiments of the invention, the concentration of the hyaluronic acid ranges from 0.01 to 0.03% w/w.

According to some embodiments of the invention, the concentration of the hyaluronic acid is about 0.02% w/w.

According to some embodiments of the invention, the concentration of the hyaluronic acid is about 0.01% w/w

The term “about” refers to ±10%.

According to some embodiments, the composition further comprises tocopheryl acetate or tocopherol or a combination thereof.

According to some embodiments of the invention, the composition comprises collodial oatmeal.

According to some embodiments the formulation further comprises hydroxyphenyl propamidobenzoic acid.

According to other embodiments of the invention, the composition further comprises bisabolol.

According to some embodiments of the invention, the concentration of the bisabolol is from about 0.01% w/w to 1% w/w. In some embodiments of the invention, the concentration of the bisabolol and is between 0.1-1% w/w.

According to some embodiments of the invention, the concentration of the bisabolol is about 0.1% w/w.

According to other embodiments of the invention, the composition further comprises Shea butter (Butyrospermum Parkii).

In some embodiments of the invention, the concentration of the Shea butter (Butyrospermum Parkii) is between 0.5 to 10% w/w.

In some embodiments of the invention, the concentration of the Shea butter is between 2 to 5% w/w. According to some embodiments of the invention, the concentration of the Shea butter is about 2% w/w. According to some embodiments, the concentration is about 5% w/w.

According to some embodiments the composition comprises vegetable oils.

In some embodiments of the invention, the concentration of the glycerin is between 1-70% w/w. In some embodiments of the invention, the concentration of the glycerin is between 4-40% w/w. In some embodiments of the invention, the concentration of the glycerin is between 5-32% w/w.

According to some embodiments of the invention, the concentration of the glycerin is about 32% w/w. According to some embodiments of the invention, the concentration of the glycerin in the gel dosage form of the invention is about 32% w/w.

According to some embodiments of the invention, the concentration of the glycerin is about 10% w/w. According to some embodiments of the invention, the concentration of the glycerin in the face cream or the body lotion/cream dosage forms of the invention, is about 10% w/w.

According to some embodiments of the invention, the concentration of the glycerin is about 5% w/w. According to some embodiments of the invention, the concentration of the glycerin in the calming lotion dosage form of the invention, is about 5% w/w.

According to other embodiments of the invention, the composition further comprises skin conditioning agents.

The term “skin conditioning agents” refers to ingredients that moisturize and strengthen the skin barrier. Skin conditioning agents which may be used include petrolatum, caprylic/capric triglyceride, dimethicone, butyrospermum parkii (shea butter), glycerin, mineral oil, persea gratissima (avocado) oil, or any combination thereof.

According to other embodiments of the invention, the composition further comprises an emulsifier and/or a viscosity increasing agent.

The term “emulsifier” or the term “viscosity increasing agent” refers to a ingredients that stabilize the emulsion. Emulsifiers and/or a viscosity increasing agents which may be used include without limitation, cetearyl alcohol, cetyl alcohol, cetearyl glucoside glyceryl stearate, PEG-100 stearate, ceteareth-33, sodium acrylate/sodium acryloyldimethyl taurate copolymer, polysorbate 80, polyacrylate 13, polyisobutene & polysorbate 20, acrylates/C10-30 alkyl acrylate crosspolymer, or any combination thereof.

The expression “dermatologically acceptable carrier” as used herein, means a carrier that is compatible with the skin, scalp, hair, nail and the like.

The topical composition of the invention is particularly useful in the treatment and/or prevention of dermatitis, especially contact dermatitis and atopic dermatitis and other skin allergies or sensitive skin.

The compositions of the invention show high dermal compatibility and low irritation behavior when applied to human skin. Further, as can be seen by the Examples, it was surprisingly found that the moisturizing level of the aforementioned formulations as was measured by Trans Epidermal Water Loss (TEWL) is significantly reduced in comparison to the level of TEWL before the treatment.

In some embodiments of the invention, the composition of the invention shows a synergistic beneficial effect on various symptoms of atopic dermatitis and/or on TEWL.

As can be seen in Examples 5, 6 and 8, the compositions prepared according to certain embodiments of the invention demonstrated dramatic therapeutic effects in atopic dermatitis patients. As can be seen, the levels of erythema and oozing, as well as, the dryness, which are characteristics of atopic dermatitis of the skin were significantly reduced following a three week treatment with an exemplary body lotion/cream composition of the invention.

These results show the superior effect of the composition of the invention on atopic dermatitis symptoms and substantiate the therapeutic and cosmetic value of the composition of the invention in atopic dermatitis as well as in other chronic inflammatory skin diseases and allergic skin diseases. Moreover, as can be seen from Example 8, the compositions of the invention, were much more effective in different parameters that were examined, such as, redness, dryness, itching feeling and the like. The results presented in this example clearly shows that the modification of the formulations contributed to higher efficacy of the product. The formulations that comprise the herbal mixture together with additional active ingredients such as hyaluronic acid, a mixture of cholesterol, ceramides and phytosphingosine, moisturizers (avocado oil, shea butter, glycerin) hydroxyphenyl propamidobenzoic acid and bisabolol improve the efficacy of the formulations and can therefore can act synergistically for atopic dermatitis/sensitive skin treatment.

Moreover, according to the results of the questionnaire the new formulations were evaluated as having a better efficacy on patients with sensitive skin/atopic dermatitis, texture and feeling in comparison to the previous formulations

Accordingly, in an embodiment of the invention, there is provided a method of treating and/or preventing a chronic inflammatory skin disease or disorder comprising the step of contacting the skin or the scalp of a subject in need with the composition of the invention.

Some embodiments of the invention are directed to the use of the composition of the invention in the manufacturing of a medicament for treating and/or preventing a chronic inflammatory skin disease or disorder.

Some embodiments of the invention are directed to the composition of the invention for use in treating and/or preventing a chronic inflammatory skin disease or disorder.

The term “preventing” refers to keeping a disease, disorder or condition from occurring in a subject. In some cases the subject may be at risk for developing the disease, but has not yet been diagnosed as having the disease. In some embodiments, the term “preventing” refers to preventing the occurrence of the next cycle of the disease.

The term “chronic inflammatory skin disease(s) or disorder(s)” include dermatitis conditions and skin impairments such as: atopic dermatitis, contact dermatitis, allergic contact dermatitis, allergic dermatitis, nummular dermatitis, chronic dermatitis of hands and feet, generalized exfoliative dermatitis, stasis dermatitis, neonatal dermatitis, pediatric dermatitis, generalized exfoliative dermatitis; stasis dermatitis; localized scratch dermatitis, toxic/irritating contact eczema, allergic contact eczema, type I or type IV, photoallergic contact eczema, contact urticaria, dyshidrosiform eczema, age-caused wrinkles, sun damage and itching, sensitive skin, senile dermatitis, radiation dermatitis, skin rashes, angioedema, eczema, irritated skin following insects bites

Other dermatological disorders which may be treated by the composition of the invention are: Psoriasis: psoriasis vulgaris, flaking eczema, Photodermatosis: radiation dermatitis, radiodermatitis acuta and chronica (UV and ionizing radiation therapy), chronic actinic dermatitis, photourticaria (urticaria solaris), polymorphic photodermatosis and other polymorphic photodermatosis; Prurigo: p. simplex acuta (strophulus, urticaria papulosa), subacuta, chronica; Deficinent ipoactive skin: localized scratch dermatitisrinophyma, ichthyosis, xerosis; Perioral dermatitis, senile dermatitis.

Using routine methods, the topical compositions of the present invention may be formulated into a variety of preparations, depending on the intended use. These preparations include, but are not limited to, topical skin compositions for medical use, topical skin cosmetic compositions, medical devices, monograph OTC and hair-treatment compositions.

As topical skin compositions for medical use and topical skin cosmetic compositions, many forms of gels, ointments, soaps, creams and lotions may be used.

When the topical compositions of the present invention are used as cosmetic compositions, cosmetic or dermatological acceptable ingredients may be optionally incorporated in arbitrary combinations as desired and determined in accordance by a person skilled in the art. According to some embodiments, the compositions may include oils, fats, waxes, surfactants, chelating agents, pH modifiers, preservatives, viscosity modifiers, colorants, perfumes, dyestuffs and the like.

In some embodiments of the invention, a preservative is added to the composition. Preservative which may be used include dehydroacetic acid, potassium sorbate, phenoxyethanol, DMDM Hydantoin and the like and any combination thereof.

The composition may contain humectants such as proteins or protein hydrolysates, amino acids, polyols, urea, allantoin, sugars and derivatives, water-soluble vitamins, plant extracts, hydroxyacids, polyols (e.g. glycerol), vitamins (e.g. D-panthenol), and/or allantoin.

The composition may be in a form of oil-in-water, water-in-oil, water-in-oil-in-water, and oil-in-water-in-silicone emulsions, a cream, an ointment, an aqueous solution, a lotion, a soap, a paste, a foam, an emulsion, a gel, a salves, an oil, a wash, a shampoos, a conditioners or an aerosol.

In an embodiment of the invention, the composition is in a form of a topical body lotion/cream and comprises Rheum palmatum Root Extract, Cnidium monnieri Fruit Extract, Scutellaria baicalensis Root Extract, dipotassium glycyrrhizate, hyaluronic acid, glycerin, cholesterol, ceramide, phytosphingosine, tocopheryl acetate, shea butter.

In an embodiment of the invention, the composition is in a form of a topical body lotion/cream and comprises Rheum palmatum Root Extract, Cnidium monnieri Fruit Extract, Scutellaria baicalensis Root Extract, dipotassium glycyrrhizate, hyaluronic acid, glycerin and one or more of mineral oil, cholesterol, ceramide, phytosphingosine, cetyl acetate, sodium lauroyl lactylate, carbomer, xanthan gum, tocopheryl acetate, shea butter, petrolatum, caprylic/capric triglyceride, dimethicone, cetearyl alcohol, cetyl alcohol, glyceryl stearate, dehydroacetic acid and potassium sorbate or any combination thereof.

In another embodiment of the invention, the composition is in a form of a face cream and comprises Rheum palmatum Root Extract, Cnidium monnieri Fruit Extract, Scutellaria baicalensis Root Extract, dipotassium glycyrrhizate, hyaluronic acid, glycerin, cholesterol, ceramide, phytosphingosine, tocopheryl acetate, hydroxyphenyl propamidobenzoic, bisabolol and shea butter,

In another embodiment of the invention, the composition is in a form of a face cream and comprises Rheum palmatum Root Extract, Cnidium monnieri Fruit Extract, Scutellaria baicalensis Root Extract, dipotassium glycyrrhizate, hyaluronic acid, glycerin, and one or more of mineral oil, persea gratissima (avocado) oil, cholesterol, ceramide, phytosphingosine, sodium lauroyl lactylate, carbomer, xanthan gum, tocopheryl acetate, hydroxyphenyl propamidobenzoic, bisabolol, shea butter, caprylic/capric triglyceride, cetearyl alcohol, cetyl alcohol, sodium acrylate/sodium acryloyldimethyl taurate copolymer, polyacrylate 13, dehydroacetic acid and potassium sorbate, or any combination thereof.

In an embodiment of the invention, the composition is in a form of a topical calming lotion and comprises Rheum palmatum Root Extract, Cnidium monnieri Fruit Extract, Scutellaria baicalensis Root Extract, dipotassium glycyrrhizate, hyaluronic acid, glycerin, hydroxyphenyl propamidobenzoic acid and bisabolol,

In an embodiment of the invention, the composition is in a form of a topical calming lotion and comprises Rheum palmatum Root Extract, Cnidium monnieri Fruit Extract, Scutellaria baicalensis Root Extract, dipotassium glycyrrhizate, hyaluronic acid, glycerin, hydroxyphenyl propamidobenzoic acid, bisabolol and one or more of caprylic/capric triglyceride, cetearyl ethylhexanoate, dimethicone, phenyl trimethicone, polyacrylate 13, acrylates/C10-30 alkyl acrylate crosspolymer, dehydroacetic acid and potassium sorbate or any combination thereof.

In an embodiment of the invention, the composition is in a form of a topical moisturizing gel and comprises Rheum palmatum Root Extract, Cnidium monnieri Fruit Extract, Scutellaria baicalensis Root Extract, dipotassium glycyrrhizate, hyaluronic acid, and glycerin.

In an embodiment of the invention, the composition is in a form of a topical moisturizing gel and comprises Rheum palmatum Root Extract, Cnidium monnieri Fruit Extract, Scutellaria baicalensis Root Extract, dipotassium glycyrrhizate, hyaluronic acid, glycerin and one or more of cyclopentasiloxane, caprylic/capric triglyceride, dimethicone, polyacrylamide, phenoxyethanol and DMDM hydantoin or any combination thereof.

In an embodiment of the invention, the composition is in a form of a body lotion/cream and comprises from 0.1% to 2% w/w Rheum palmatum Root Extract, from 0.1% to 2% w/w Cnidium monnieri Fruit Extract, from 0.1% to 2% w/w Scutellaria baicalensis Root Extract, from 0.1% to 2% w/w dipotassium glycyrrhizate, from 0.001 to 0.2% w/w hyaluronic acid, from 5% to 20% w/w glycerin, from 1% to 10% w/w mineral oil, from 0.005% to 1% w/w cholesterol, from 0.005% to 1% w/w ceramide and from 0.005% to 1% w/w phytosphingosine, from 0.05% to 1% w/w tocopheryl acetate, from 0.5 to 10% w/w shea butter, from 5% to 20% w/w petrolatum, from 1% to 10% w/w caprylic/capric triglyceride. The composition may comprise dimethicone, cetearyl alcohol, cetyl alcohol, from glyceryl stearate, dehydroacetic acid and potassium sorbate.

It is noted that the percentage of the herbal extracts (Rheum palmatum, Scutellaria baicalensis or Cnidium monnieri) are calculated when using extracts characterized as dark brown sticky liquids having a density at 25° C. of 1-1.1 g/ml. If different herbs preparations are used, the percentage should be calculated on the basis of the new density.

In other embodiments of the invention, the composition is in a form of a topical face cream and comprises 1% to 2% w/w Rheum palmatum Root Extract, from 0.1% to 2% w/w Cnidium monnieri Fruit Extract, from 0.1% to 2% w/w Scutellaria baicalensis Root Extract, from 0.1% to 2% w/w dipotassium glycyrrhizate, from 0.001 to 0.2% w/w hyaluronic acid, from 1% to 20% w/w glycerin, from 1% to 10% w/w mineral oil, from 0.5% to 5% w/w persea gratissima (avocado) oil, from 0.005% to 1% w/w cholesterol, from 0.005% to 1% w/w ceramide, from 0.005% to 1% w/w phytosphingosine, from 0.05% to 1% w/w tocopheryl acetate, from 0.001% to 1% w/w hydroxyphenyl propamidobenzoic acid, from 0.05% to 1% w/w bisabolol and from 0.5 to 10% w/w shea butter. In some embodiments, the composition may further comprise sodium lauroyl lactylate. Carbomerand xanthan gum. According to some further embodiments, the formulation may further comprise caprylic/capric triglyceride, cetearyl alcohol, cetyl alcohol, sodium acrylate/sodium acryloyldimethyl taurate copolymer, polyacrylate 13, dehydroacetic acid, and potassium sorbet. In some embodiments, the composition may further comprise sodium lauroyl lactylate. Carbomerand xanthan gum.

In other embodiments of the invention, the composition is in a form of a topical calming lotion and comprises 1% to 2% w/w Rheum palmatum Root Extract, from 0.1% to 2% w/w Cnidium monnieri Fruit Extract, from 0.1% to 2% w/w Scutellaria baicalensis Root Extract, from 0.1% to 2% w/w dipotassium glycyrrhizate, from 0.001 to 0.2% w/w hyaluronic acid, from 1% to 70% w/w glycerin, from 0.001% to 1% w/w hydroxyphenyl propamidobenzoic acid and from 0.01% to 1% w/w bisabolol. In some embodiments, the formulation may further comprisecaprylic/capric triglyceride, cetearyl ethylhexanoate, dimethicone, from phenyl trimethicone, polyacrylate 13, from acrylates/C10-30 alkyl acrylate crosspolymer, from dehydroacetic acid, and potassium sorbate.

In an embodiment of the invention, the composition is in a form of a topical moisturizing gel and comprises the following 0.1% to 2% w/w Rheum palmatum Root Extract, from 0.1% to 2% w/w Cnidium monnieri Fruit Extract, from 0.1% to 2% w/w Scutellaria baicalensis Root Extract, from 0.1% w/w to 2% w/w dipotassium glycyrrhizate, from 0.001 to 0.2% w/w hyaluronic acid, from 1% to 50% w/w glycerin. The formulation may in some embodiments further comprise one or more of cyclopentasiloxane, caprylic/capric triglyceride, dimethicone, polyacrylamide, phenoxyethanol and DMDM hydantoin and any combination thereof.

In an embodiment of the invention, the composition is in a form of a body lotion/cream and comprises the following about 1% w/w Rheum palmatum Root Extract, about 1% w/w Cnidium monnieri Fruit Extract, about 1% w/w Scutellaria baicalensis Root Extract, about 1% w/w dipotassium glycyrrhizate, about 0.02% w/w hyaluronic acid, about 10% w/w glycerin, about 5% w/w mineral oil, about 0.01% w/w cholesterol, about 0.03% w/w ceramides, about 0.01% w/w phytosphingosine, about 1.5% w/w cetyl acetate, about 0.2% w/w sodium lauroyl lactylate, about 0.06% w/w carbomer, about 0.06% w/w xanthan gum, about 0.1% w/w tocopheryl acetate, about 5% w/w shea butter, about 15% w/w petrolatum about 4% w/w caprylic/capric triglyceride, about 0.5% w/w dimethicone, about 4.8% w/w cetearyl alcohol, about 3% w/w cetyl alcohol, from 0.25% w/w to 0.5% w/w glyceryl stearate, about 0.008-0.08% w/w dehydroacetic acid and about 0.2% w/w potassium sorbate, In other embodiments of the invention, the composition is in a form of a face cream and comprises about 1% w/w Rheum palmatum Root Extract, about 1% w/w Cnidium monnieri Fruit Extract, about 1% w/w Scutellaria baicalensis Root Extract, about 1% w/w dipotassium glycyrrhizate, about 0.02% w/w hyaluronic acid, about 10% w/w glycerin, about 7.55% w/w mineral oil, about 2% w/w persea gratissima (avocado) oil, about 0.01% w/w cholesterol, about 0.03% w/w ceramides, about 0.01% w/w phytosphingosine, about 0.2% w/w sodium lauroyl lactylate, about 0.08% w/w carbomer, about 0.06% w/w xanthan gum, about 0.2% w/w tocopheryl acetate, about 0.005-0.025% w/w hydroxyphenyl propamidobenzoic, about 0.1% w/w bisabolol, about 2% w/w shea butter, about 4% w/w caprylic/capric triglyceride, about 3-3.4% w/w cetearyl alcohol, about 2.5% w/w cetyl alcohol, about 0.6% w/w sodium acrylate/sodium acryloyldimethyl taurate copolymer, about 0.6% w/w polyacrylate 13, about 0.008-0.08% w/w dehydroacetic acid, and about 0.2% w/w potassium sorbate.

In other embodiments of the invention, the composition is in a form of a calming lotion and comprises about 1% w/w Rheum palmatum Root Extract, about 1% w/w Cnidium monnieri Fruit Extract, about 1% w/w Scutellaria baicalensis Root Extract, about 1% w/w dipotassium glycyrrhizate, about 0.02% w/w hyaluronic acid, about 5% w/w glycerin, about 0.005-0.025% w/w hydroxyphenyl propamidobenzoic, about 0.1% w/w bisabolol, about 4% w/w caprylic/capric triglyceride, 2.25-2.5 X % w/w dimethicone, about 1% w/w phenyl trimethicone, about 1.2% w/w polyacrylate 13, about 0.3% w/w acrylates/C10-30 alkyl acrylate crosspolymer, about 0.008-0.08% w/w dehydroacetic acid and about 0.2% w/w potassium sorbate,

In other embodiments of the invention, the composition is in a form of a Extra moisturizing gel and comprises the following about 1% w/w Rheum palmatum Root Extract, about 1% w/w Cnidium monnieri Fruit Extract, about 1% w/w Scutellaria baicalensis Root Extract, about 1% w/w dipotassium glycyrrhizate, about 0.01% w/w hyaluronic acid, about 32% w/w glycerin, about 23.24-23.80% w/w cyclopentasiloxane, about 1% w/w caprylic/capric triglyceride, about 3.64-4.76% w/w dimethicononol, about 1.68% w/w polyacrylamide, about 0.708-0.732% w/w phenoxyethanol and about 0.35% w/w DMDM hydantoin.

According to an embodiment of the invention, the compositions of the invention are administered once a day. According to other embodiments, the compositions are administered twice a day, three times a day or more.

According to an embodiment of the invention, the composition is administered chronically.

According to some embodiments of the invention, the composition is administered for about 10 days or more, 20 days, 30 days, 60 days, 90, 120 days or more.

The CTFA Cosmetic Ingredient Handbook, Second Edition (1992) describes a wide variety of non-limiting cosmetic ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizate), skin treating agents, thickeners, and vitamins and derivatives thereof.

The plant extracts used herein may be purified by the use of a polar solvent (i.e. polar extract) such as, ethyl alcohol (ethanol), butyl alcohol (butanol), methanol, water or propanol. The polar extracts of the present invention may comprise any percentage of a polar solvent.

Alternatively, the plant extracts may be purified by the use of a non-polar solvent (i.e. non-polar extract) such as, without being limited to, isooctane. The non-polar extracts of the present invention may comprise any percentage of non-polar solvents.

Typically, hydrophobic molecules tend to be non-polar and thus non-polar solvents are used. Hydrophilic molecules tend to be polar and dissolve by water and/or other polar substances.

The active ingredients can be concentrated in the extract via affinity chromatography, mass chromatography and the like.

Typically, the plant extract of the present invention is an aqueous extract. In order to obtain a purified plant extract (e.g. with reduced levels of organic salts and/or heavy metals and/or starch in the plant extract), the aqueous plant extract is typically further purified using a resin chromatography such as a macroporous resin or other chromatography methods.

Thus, according to another embodiment, there is provided a method of preparing a composition for preparing concentrated herbal extracts for treating and/or preventing atopic dermatitis, other allergic skin diseases and skin infections, the method comprising: (a) subjecting a plant to x 1-10 volumes of water to produce an extract of the plant; and (b) reducing the amount of organic salts and/or heavy metals and/or starch in the plant extract using a macroporous resin which results in an elevated content of the active ingredients present in the plant extract.

### EXAMPLES

### Example 1

TABLE 1
Comparison of exemplary new and previous Topical Body Lotion formulations:

| Formulation 1 (New) | | Formulation 2 (Previous) | |
|---|---|---|---|
| INCI Name | Function | INCI Name | Function |
| Aqua | Solvent | Aqua | Solvent |
| Petrolatum | Skin conditioning | Petrolatum | Skin conditioning |
| | agent | | agent |
| Glycerin | Moisturizer | Glycerin | Moisturizer |
| Cetyl Acetate | Emollient | Cetyl Acetate | Emollient |
| Caprylic/Capric | Skin conditioning | Caprylic/Capric | Skin Calmer |
| Triglyceride | agent | Triglyceride | |
| Cetearyl Alcohol | Emulsifier | Cetearyl Alcohol | Viscosity Builder |
| Cetyl Alcohol | Emulsifying agent | Cetyl Alcohol | Viscosity Builder |
| | Viscosity increasing | | |
| | agent | | |
| Stearyl Acetate | | Stearyl Acetate | |
| Oleyl Acetate | | Oleyl Acetate | |
| Cholesterol | | Cholesterol | Emollient |
| Acetylated Lanolin | | Acetylated Lanolin | Emollient |
| Alcohol | | Alcohol | |
| Cetearyl | Skin conditioning | Cetearyl | Emollient |
| Ethylhexanoate | agent | Ethylhexanoate | |
| Dipotassium | Skin Calmer | Dipotassium | Skin Calmer |
| Glycyrrhizate | | Glycyrrhizate | |
| Scutellaria | | Scutellaria | Active Botanical |
| Baicalensis Root | | Baicalensis Root | extracts |
| Extract | | Extract | |
| Rheum Palmatum | Active botanicals | Rheum Palmatum | |
| Root Extract | extracts | Root Extract | |
| Cnidium Monnieri | | Cnidium Monnieri | |
| Fruit Extract | | Fruit Extract | |
| Dimethicone | Skin conditioning | Dimethicone | Antifoam |
| | agent | | |
| Tocopheryl Acetate | Antioxidant | Tocopheryl Acetate | Antioxidant |
| Butyrospermum | Skin conditioning | Paraffinum | Emollient |
| Parkii (Shea Butter) | agent | Liquidum | |
| Mineral Oil | Skin conditioning | Lanolin Alcohol | Moisturizer |
| | agent | | |
| Glyceryl Stearate | Emulsifier | Ceteareth -20 | Emulsifier |
| Dehydroacetic Acid | Preservative Mixture | Methylparaben | Preservative |
| Potassium sorbate | Preservative | Imidazolidinyl Urea | Preservative |
| Cetearyl Glucoside | | Propylparaben | Preservative |
| Ceramide 3 | Skin repair active | Laminaria | |
| | | Ochroleuca Extract | |
| Ceramide 6 II | | Rosa Damascena | Botanical Additive |
| | | Flower Water | |
| Ceramide 1 | | | |
| Phytosphingosine | | | |
| Cetearyl Alcohol | Viscosity increasing | | |
| | agent | | |
| Sodium Lauroyl | | | |
| Lactylate | | | |
| Carbomer | | | |
| PEG-100 Stearate | | | |
| Benzyl Alcohol | | | |
| Sucrose Stearate | | | |
| Pentadecalactone | Fragrance mixture | | |
| Triethyl Citrate | | | |
| Xanthan Gum | Viscosity increasing | | |
| | agent | | |
| Lactic Acid | pH adjuster | | |
| Hyaluronic Acid | Skin conditioning | | |
| | agent | | |

The new formulation is different from the previous one in at least the following aspects: Antilukine 6 (Laminaria ochroleuca Extract) was replaced by hyaluronic acid (HA) as an active ingredient contributes to the enhancement of skin hydration and reduction of skin dryness.

Cholesterol in the previous formulation was replaced by a mixture of cholesterol and ceramides (Ceramide 3 & Ceramide 6 II & Ceramide 1 & Phytosphingosine) in the new formulation.

### Example 2

TABLE 2
Comparison of exemplary new and previous Topical Face Cream formulations:

| Formulation 3 (New) | | Formulation 4 (Previous) | |
|---|---|---|---|
| INCI Name | Function | INCI Name | Function |
| Aqua | Solvent | Aqua | Solvent |
| Glycerin | Moisturizer | Glycerin | Moisturizer |
| Caprylic/Capric | Skin conditioning | Caprylic/Capric | Skin Calmer |
| Triglyceride | agent | Triglyceride | |
| Cetearyl Alcohol | Emulsifier | Cetearyl Alcohol | Self Emulsified |
| | | | Wax |
| Cetyl Alcohol | Emulsifying agent | Cetyl Alcohol | Viscosity enhancer |
| | Viscosity increasing | | |
| | agent | | |
| Cholesterol | | Cholesterol | Emollient |
| Dipotassium | Skin Calmer | Dipotassium | Skin Calmer |
| Glycyrrhizate | | Glycyrrhizate | |
| Scutellaria | | Scutellaria | |
| Baicalensis Root | | Baicalensis Root | |
| Extract | | Extract | |
| Rheum Palmatum | Active botanicals | Rheum Palmatum | Botanical Additives |
| Root Extract | extracts | Root Extract | |
| Cnidium Monnieri | | Cnidium Monnieri | |
| Fruit Extract | | Fruit Extract | |
| Tocopheryl Acetate | Antioxidant | Tocopheryl Acetate | Anti Oxidant (Vit. E) |
| Ceteareth-33 | | Ceteareth 33 | |
| Ceramide 3 | Skin repair active | Ceramide-3 | Active, Conditioner |
| Sodium Acrylate/ | Thickening | Sodium Acrylate/ | Stabilizer |
| Sodium | emulsifying agent | Acryloyldimethy | |
| Acryloyldimethyl | | | |
| Taurate Copolymer | | | |
| Butyrospermum | Skin conditioning | Butyrospermum | Emollient |
| Parkii (Shea Butter) | agent | Parkii Fruit | |
| Isohexadecane | | Isohexadecane | |
| Polysorbate 80 | | Polysorbate 80 | |
| Dehydroacetic Acid | Preservative Mixture | Methylparaben | Preservatives |
| | | | Mixture |
| | | Ethylparaben | |
| | | Butylparaben | |
| | | Propylparaben | |
| | | Isobutylparaben | |
| Potassium sorbate | Preservative | Methylchloroisothiazoli- | Preservatives |
| | | none | Mixture |
| Mineral Oil | Skin conditioning | Methylisothiazolinone | |
| | agent | | |
| Ceramide 6 II | | Paraffinum Liquidum | Emollient |
| Ceramide 1 | | PEG-8 | Moisturizer |
| Phytosphingosine | | Laminaria Ochroleuca | |
| | | Extract | |
| Sodium Lauroyl | | Borago Officinalis | Light Emollient |
| Lactylate | | Seed Oil | |
| Carbomer | Carbomer | Phenoxyethanol | |
| Xanthan Gum | | | |
| Persea Gratissima | Skin conditioning | | |
| (Avocado) Oil | agent | | |
| Polyacrylate 13 | Emulsifier | | |
| Polyisobutene | | | |
| Polysorbate 20 | | | |
| Benzyl Alcohol | | | |
| Butylene Glycol | Skin Calmer mixture | | |
| Pentylene Glycol | | | |
| Hydroxyphenyl | | | |
| Propamidobenzoic | | | |
| Acid | | | |
| Pentadecalactone | Fragrance mixture | | |
| Triethyl Citrate | | | |
| Bisabolol | Skin Calmer | | |
| Lactic Acid | pH adjuster | | |
| Hyaluronic Acid | Skin conditioning | | |
| | agent | | |

Antilukine 6 (laminaria ochroleuca Extract) in the previous formulation was replaced by with hyaluronic acid (HA) in the new formulation (3).

Cholesterol and ceramide 3 (in the previous formulation) was replaced by a mixture of cholesterol and ceramides (Ceramide 3 & Ceramide 6 II & Ceramide 1 & phytosphingosine) in the new formulation (3).

Further, the new formulation (3) contains additional moisturizers, such as, avocado oil, higher concentration of shea butter, which replace the borago oil, and contains additional anti-irritant ingredients such as hydroxyphenyl propamidobenzoic acid and bisabolol.

### Example 3

TABLE 3
Comparison of exemplary new and previous Topical Calming Lotion formulations

| Formulation 5 (New) | | Formulation 6 (Previous) | |
|---|---|---|---|
| INCI Name | Function | INCI Name | Function |
| Aqua | Solvent | Aqua | Solvent |
| Glycerin | Moisturizer | Glycerin | Moisturizer |
| Dimethicone | | Dimethicone | |
| Cyclopentasiloxane | Cooling & Mattifying | Cyclotetrasiloxane | Cooling & Mattifying |
| | Agents | | Agents |
| Polysilicone-11 | | Polysilicone-11 | |
| Caprylic/Capric | Skin calmer | Caprylic/Capric | Skin calmer |
| Triglyceride | | Triglyceride | |
| Rheum Palmatum | Active botanicals | Rheum Palmatum | Active botanicals |
| Root Extract | extracts | Root Extract | extracts |
| Cnidium Monnieri | | Cnidium Monnieri | |
| Fruit Extract | | Fruit Extract | |
| Scutellaria | | Scutellaria | |
| Baicalensis Root | | Baicalensis Root | |
| Extract | | Extract | |
| | | Polysorbate-20 | Emulsifier |
| Dipotassium | Skin Calmer | Dipotassium | Skin Calmer |
| Glycyrrhizate | | Glycyrrhizate | |
| Phenyl Trimethicone | Skin conditioning | Phenyl Trimethicone | Light Emollient |
| | agent | | |
| Triethanolamine | PH Adjuster | Triethanolamine | PH Adjuster |
| Acrylates/C10-30 | Emulsifier& Stabilizer | Acrylates/C10-30 | Emulsifier& Stabilizer |
| Alkyl Acrylate | | Alkyl Acrylate | |
| Crosspolymer | | Crosspolymer | |
| Polyacrylate 13 | Emulsifier | Laminaria | |
| | | Ochroleuca Extract | |
| Polyisobutene | | Phenoxyethanol | Preservative Mixture |
| Dehydroacetic Acid | Preservative Mixture | Methylparaben | |
| Benzyl Alcohol | | Ethylparaben | |
| Butylene Glycol | Skin Calmer mixture | Butylparaben | |
| Pentylene Glycol | | Propylparaben | |
| Hydroxyphenyl | | Isobutylparaben | |
| Propamidobenzoic | | | |
| Acid | | | |
| Potassium Sorbate | Preservative | Polyacrylamide | Gel Former & |
| | | | Stabilizer |
| Pentadecalactone | Fragrance mixture | C13-14 Isoparaffin | |
| Triethyl Citrate | | Laureth-7 | |
| Polysorbate 20 | Stabilizer | Tetrasodium EDTA | Water Softener |
| Bisabolol | Skin Calmer | | |
| Disodium EDTA | Chelating agent | | |
| Hyaluronic Acid | Skin conditioning | | |
| | agent | | |

Antilukine 6 (laminaria ochroleuca Extract) in the previous formulation was replaced by hyaluronic acid (HA) in the new formulation (5).

Further, the new formulation (5) contains additional anti-irritant ingredients such as hydroxyphenyl propamidobenzoic acid and bisabolol which replace the guaiazulene.

### Example 4

TABLE 4
Comparison of exemplary new and previous Topical Moisturizing Gel formulations

| Formulation 7 (New) | | Formulation 8 (Previous) | |
|---|---|---|---|
| INCI Name | Function | INCI Name | Function |
| Aqua | Solvent | Aqua | Solvent |
| Glycerin | Moisturizer | Glycerin | Moisturizer |
| Cyclopentasiloxane | Emollient | Cyclotetrasiloxane | Emollient |
| Dimethiconol | | Dimethicone | Emollient |
| Polyacrylamide | Emulsifier | Polyacrylamide | |
| C13-14 Isoparaffin | | C13-14 Isoparaffin | Gel Former |
| Laureth-7 | | Laureth - 7 | |
| Dipotassium | Skin Calmer | Dipotassium | Skin Calmer |
| Glycyrrhizate | | Glycyrrhizate | |
| Scutellaria Baicalensis | Active Botanical | Scutellaria Baicalensis | |
| Root Extract | Extract | Root Extract | |
| Rheum Palmatum | Active Botanical | Rheum Palmatum | Active Botanical |
| Root Extract | Extract | Root Extract | Extracts |
| Cnidium Monnieri Fruit | Active Botanical | Cnidium Monnieri Fruit | |
| Extract | Extract | Extract | |
| Caprylic/Capric | Skin calmer | Caprylic/Capric | Skin Calmer |
| Triglyceride | | Triglyceride | |
| Ethoxydiglycol | Moisturizer | Ethoxydiglycol | Moisturizer |
| Phenoxyethanol | Preservative | Sodium | Preservative |
| | | Methylparaben | |
| DMDM Hydantoin | Preservative | Laminaria Ochroleuca | |
| | | Extract | |
| Ethylhexylglycerin | | Rosa Damascena | Perfume |
| | | Flower Water | |
| Pentadecalactone | Fragrance mixture | Guaiazulene | Skin Calmer + Color |
| Triethyl Citrate | | | |
| Hyaluronic Acid | Skin conditioning | | |
| | agent | | |

Antilukine 6 (laminaria ochroleuca Extract) in the previous formulation was replaced by hyaluronic acid (HA) in the new formulation (7).

The resulted new formulations in Examples 1-4 are characterized as follows: TOPIC Body lotion/Cream—a very thick cream, yellowish; Topic Face Cream—thick cream, white—yellowish; TOPIC Calming Lotion—yellowish brown thick liquid; and TOPIC Topical Moisturizing Gel—thick gel, orange brown, respectively.

### Example 5

### Evaluation of the Trans Epidermal Water Loss (TWEL) Following Administration of Formulation 1 (Topical Body Lotion—New Formulation) Described in Example 1

### Method

The transepidermal water loss (TEWL) of the skin is one of the most important parameter to analyze the barrier function of the skin. TEWL is defined as the evaporation of water from the body through the skin, excluding the water loss caused by sweating. Atopic dermatitis patients have high TEWL that results in very dry skin. A preparation that upholds or even improves the skin barrier function also causes a TEWL-reduction. Low TEWL-values reflect a low evaporation of water through the skin and a good water balance inside the skin

The quantitative analysis of transepidermal water loss (TEWL) of the skin is done using an Evaporimeter. The measuring sensor of the Evaporimeter contains a duct, which is open at top side. This duct contains two sensors to measure the skin's humidity and two sensors to measure the skin's temperature. Temperature and humidity measuring sensor units are located on top of each other separated only by a little space between them. The humidity measuring sensors detect the vapour pressure in the diffusion zone directly over the skin.

With computer support the water is measured in g/hm² according to

Fick'sches Law of diffusion:

$$\frac{d m}{d t} = - D * A * \frac{d c}{d x}$$

dm/dt=diffusion flow
D=coefficient

### A=Area

Dc/dx=density gradient

### Procedure

The Evaporimeter from DermaLab Systems Fa. Cortex Technologies was used for all studies of trans epidermal water loss of the skin.

The measuring sensor was placed on top of the skin surface without causing extra pressure before the measuring procedure was initiated. This was done in a way that the outer rim of the Teflon-duct fully enclosed the skin area. The measurements were performed in series of measurement per test skin area including 20 cycles each. All single data from this series of measurements were automatically collected and the mean value was automatically calculated.

20 subjects, both male and female, between the ages 19 and 67 years were tested. The test areas were squares of 3 cm diagonal length on the body.

Skin measurement values were taken at three different locations on the body. The recorded values were averaged.

The test product was applied for a period of three weeks. The measurements with the Evaporimeter were performed before and after the three weeks of product application.

The product Atopic Body Lotion was applied on the body.

The water loss was measured and analyzed for each test person in g/hm2. The results are presented in the tables.

### Inclusion Criteria

The subjects were chosen to be persons with slight to moderate manifestation of atopic dermatitis as defined by pruritus, eczemateous, lichenification or xerosis in the test area.

### Exclusion Criteria

Subjects having severe or chronic skin inflammation, serious inner or chronic diseases, or subjects that intake drugs that possibly can interfere with skin reactions (Glucocorticoids, antiallergics, topical immuno modulator, etc.), or apply pharmaceutical products and skin care products with active ingredients until 7-10 days before testing, that have severe allergies or occurred severe side effects after usage of cosmetic products were not tested. Further, subjects who used a

Sunbath or a tanning bed during the study period, are known to have cancer, be pregnant or nursing, were also not tested.

### Dermatological Examinations

Before beginning the application test: All participants suffered from slight to moderate manifestation of atopic dermatitis as defined by pruritus, eczemateous, lichenification or xerosis in the test area.

During the study: No complaint of any pathological skin disorder was reported during the course of this three-week application test. Interruptions of the application test and/or medical intervention were not necessary.

After the end of the application test: During the final dermatological examination after the end of the study, none of the 20 participants showed development of any pathological skin disorder in the test area. The mentioned product was well-tolerated and did not lead to any unwanted skin reaction.

### Results and Analysis

Tables 5-6 below contain the average measured TEWL values for each subject (in g/hm2) that was measured on the test areas before and after three weeks of application. Negative values were calculated if the TEWL was reduced during the testing procedure.

TABLE 5
Presentation of the averaged measurements (TEWL-
values) taken from the 20 subjects before and after
three weeks of application in the test area.

| Subject | Before | after 3 weeks | difference | [%]change |
|---|---|---|---|---|
| 1. | 5.33 | 4.80 | −0.53 | −10.00 |
| 2. | 8.67 | 7.60 | −1.07 | −12.31 |
| 3. | 10.73 | 9.90 | −0.83 | −7.76 |
| 4. | 7.50 | 6.70 | −0.80 | −10.67 |
| 5. | 6.93 | 5.17 | −1.77 | −25.48 |
| 6. | 6.20 | 5.63 | −0.57 | −9.14 |
| 7. | 9.97 | 8.17 | −1.80 | −18.06 |
| 8. | 8.83 | 8.13 | −0.70 | −7.92 |
| 9. | 8.87 | 7.90 | −0.97 | −10.90 |
| 10. | 5.60 | 4.63 | −0.97 | −17.26 |
| 11. | 9.53 | 7.93 | −1.60 | −16.78 |
| 12. | 8.53 | 7.27 | −1.27 | −14.84 |
| 13. | 7.67 | 5.97 | −1.70 | −22.17 |
| 14. | 12.37 | 11.13 | −1.23 | −9.97 |
| 15. | 9.10 | 8.23 | −0.87 | −9.52 |
| 16. | 14.10 | 11.43 | −2.67 | −18.91 |
| 17. | 7.67 | 7.20 | −0.47 | −6.09 |
| 18. | 5.63 | 5.17 | −0.47 | −8.28 |
| 19. | 11.93 | 10.00 | −1.93 | −16.20 |
| 20. | 7.43 | 4.97 | −2.47 | −33.18 |
| Average | 8.63 | 7.40 | −1.23 | −14.29 |
| Minimum | 5.33 | 4.63 | −2.67 | −33.18 |
| Maximum | 14.10 | 11.43 | −0.47 | −6.09 |
| Stend. dev. | 2.35 | 2.07 | 0.65 | 6.88 |
| Variance | 5.50 | 4.30 | 0.42 | 47.38 |

The relative average TEWL-value (%) was calculated from all measured values obtained from each subject. The absolute differences between the calculated average TEWL-values indicates a change in TEWL capacity due to the applied preparation. As shown in Table 5, the average difference, which describes the decrease of water loss after three weeks of product application, was 1.23. Compared to the TEWL baseline, the results obtained from the treated skin showed a decrease of TEWL that amounted to 14.29%.

TABLE 6
Presentation of the averaged measurements (TEWL-values)
taken from 20 subjects from the untreated skin before
and after three weeks in the control area.

| subject | before | after 3 weeks | difference | [%]change |
|---|---|---|---|---|
| 1. | 5.77 | 5.47 | −0.30 | −5.20 |
| 2. | 7.57 | 7.50 | −0.07 | −0.88 |
| 3. | 11.63 | 11.50 | −0.13 | −1.15 |
| 4. | 8.67 | 9.57 | 0.90 | 10.38 |
| 5. | 5.90 | 6.70 | 0.80 | 13.56 |
| 6. | 6.50 | 7.60 | 1.10 | 16.92 |
| 7. | 10.20 | 9.58 | −0.62 | −6.11 |
| 8. | 9.60 | 9.87 | 0.27 | 2.78 |
| 9. | 8.33 | 8.70 | 0.37 | 4.40 |
| 10. | 5.50 | 5.80 | 0.30 | 5.45 |
| 11. | 8.20 | 7.53 | −0.67 | −8.13 |
| 12. | 8.43 | 7.77 | −0.67 | −7.91 |
| 13. | 5.87 | 5.50 | −0.37 | −6.25 |
| 14. | 10.83 | 10.30 | −0.53 | −4.92 |
| 15. | 7.73 | 7.37 | −0.37 | −4.74 |
| 16. | 12.30 | 11.20 | −1.10 | −8.94 |
| 17. | 7.27 | 7.27 | 0.00 | 0.00 |
| 18. | 6.93 | 7.80 | 0.87 | 12.50 |
| 19. | 12.37 | 11.80 | −0.57 | −4.58 |
| 20. | 6.93 | 6.67 | −0.27 | −3.85 |
| Average | 8.33 | 8.27 | −0.06 | −0.63 |
| Minimum | 5.50 | 5.47 | −1.10 | −8.94 |
| Maximum | 12.37 | 11.80 | 1.10 | 16.92 |
| Stend. dev. | 2.18 | 1.95 | 0.62 | 7.90 |
| Variance | 4.76 | 3.80 | 0.38 | 62.49 |

As shown in Table 6, in the untreated area a TEWL change of 0.63% was measured.

TABLE 7
CONCLUDING EVALUATION OF
THE TEWL MEASUREMENTS

| | TEWL value | | TEWL value in % |
|---|---|---|---|
| | in % with | Control/background | without |
| | background | in % | background |
| Atopic Body | −14.29 | −0.63 | −13.66 |
| Lotion | | | |

### Summarizing Assessment

All of the 20 study participants, suffering from atopic dermatitis, tolerated the product Atopic Body Lotion very well during the course of the three-week application test under dermatological and clinical criteria. There were no undesired or pathological skin reactions in the test area.

TEWL was measured on 20 subjects to determine the effect of the preparation Atopic Body Lotion on transepidermal water loss. The TEWL-values were obtained using the DermaLab Systems (Firma Cortex Technologies).

The change in the transepidermal water loss value was determined in the application area before and after using the preparation for a period of three weeks as well as in an untreated control area. In the treated area an improvement in TEWL-values of about 14.29% was measured. The average change in TEWL-values in the untreated control area amounted to 0.63%.

### Example 6

### Assessment of Skin Condition in 20 Atopic Dermatitis Patients FOLLOWING Treatment with Formulation 1 (Topical Body Lotion—New Formulation) Described in Example 1

Twenty atopic dermatitis patients were treated with the new formulation (formulation 1) described in Example 1 for 21 days, twice a day.

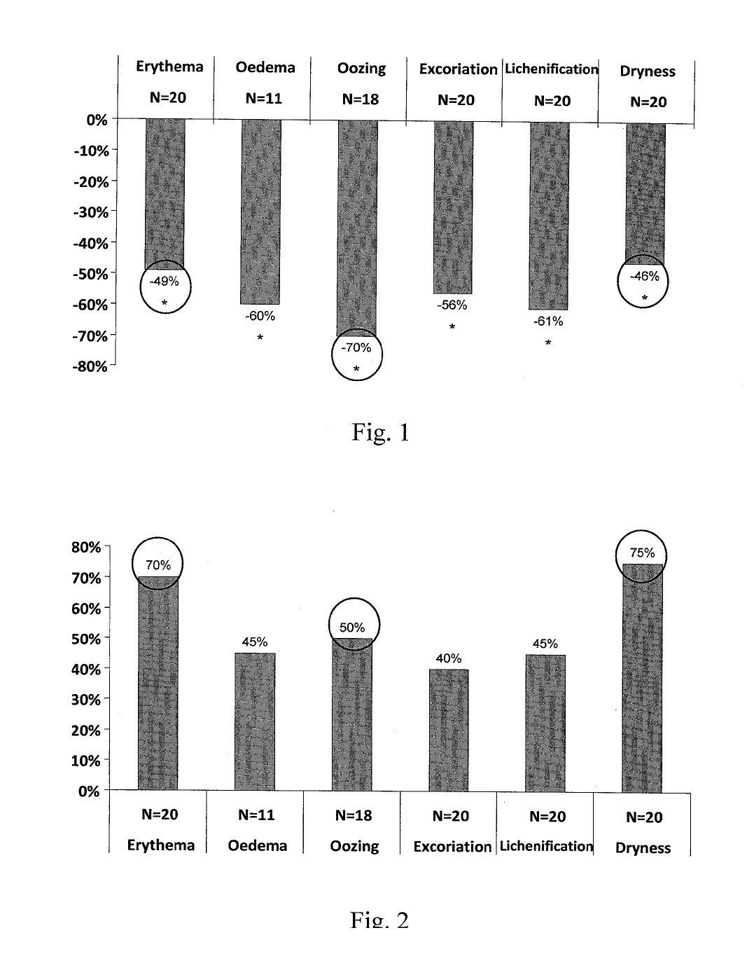

As can be seen from FIGS. 1 and 2, dryness reduction of 46% was observed in 75% of the patients, erythema reduction of 49% was observed in 70% of the patients and oozing reduction of 70% w/w was observed in 50% of the patients.

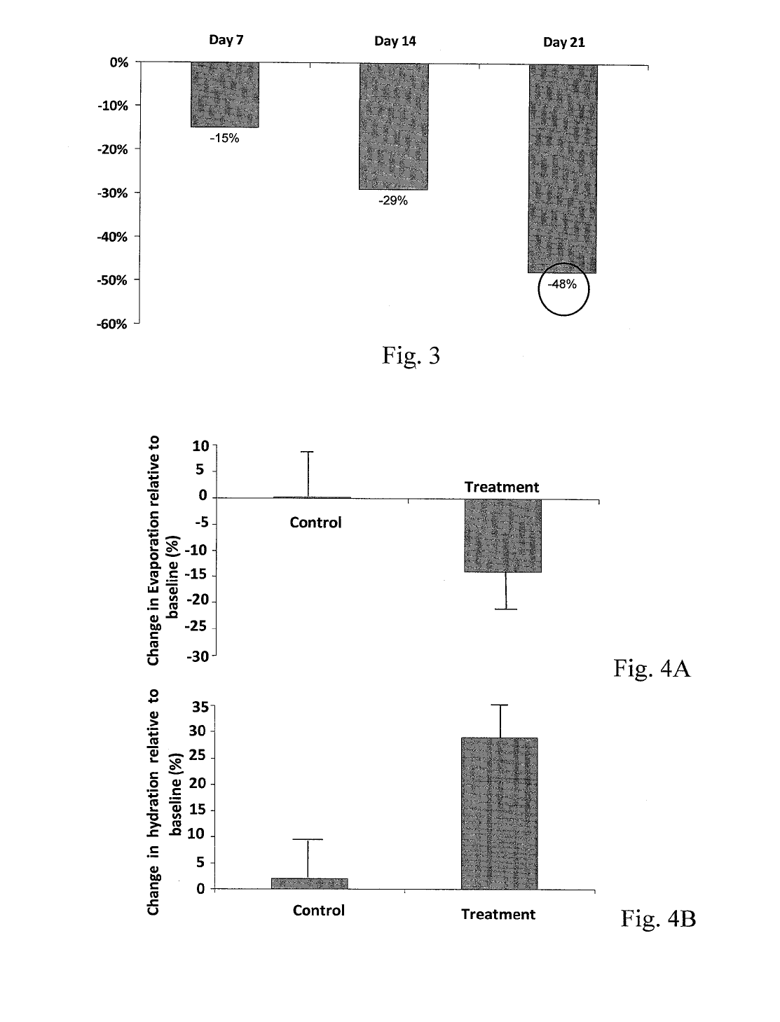

In addition, as can be seen from FIG. 3, significant reduction of the itching level was observed after 21 days of treatment.

Further, no irritation was determined by using the formulation in Patch test.

Effect of treatment on individual subjective symptoms. For each subjective symptom, only patients who expressed the symptom at baseline were analyzed. FIG. 4 shows the trans-epidermal water loss (TEWL, or evaporation) and skin hydration at baseline and after 21 days. In treated skin there was a significant reduction in TEWL/evaporation (−14±7%, p<0.001) and a significant increase in hydration (29±7%, p<0.001). In control skin no changes were observed in both measures.

### Example 7

### Assessment of Skin Condition in Atopic Dermatitis Patients Following Treatment with Topic Medis Extra Moisturizing Gel—New Formulation, Formulation 7 Described in Example 4

Subjects or parents of babies/children suffering from atopic dermatitis were instructed to apply TOPIC Medis Extra Moisturizing Gel as described in Example 4. TOPIC Medis Extra Moisturizing Gel was applied once a day for five days to the damaged areas.

Basic demographic data was collected: age, sex, initials as well as the atopic dermatitis condition: condition duration, damaged areas, other treatments.

Evaluation questionnaire for TOPIC Medis Extra Moisturizing Gel included the following questions:

Effect of the product on inflammation

Effect of the product on dryness

Effect of the product on redness

Questions 1-3 were evaluated as follows: 1—strongly aggravated, 2—aggravated, 3—no effect, 4—improved, 5—strongly improved.

### Results

Table 8 below shows demographic and condition data of the subjects.

TABLE 8
demographic and condition data of the subjects

| Initials | E M | A A | M B | T |
|---|---|---|---|---|
| Age (years) | 2 | 13 months | 65 | 11 |
| Sex | Female | Male | Female | Male |
| Duration | 2 | 13 months | 65 | 11 |
| Other | Betacorten G | A-derma, | A-derma | Dermacol |
| treatments | cream | Uriage, Dexeril, | | |
| | | Elidel | | |
| Frequency of | All the time | Once | No | Once |
| steroidal | | | | |
| treatment | | | | |

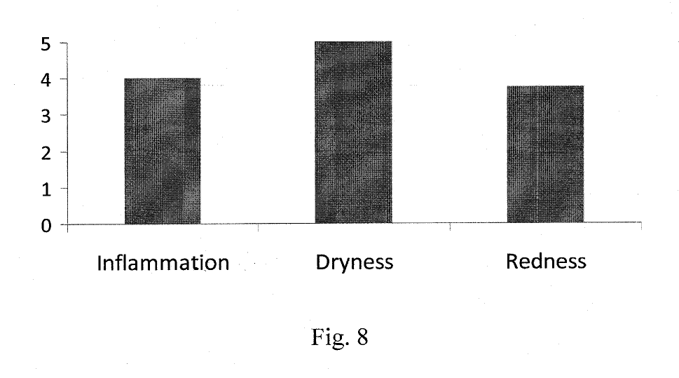

FIG. 8 shows an evaluation of the three major symptoms by the subjects or their parents (for babies). As can be seen from FIG. 8 a marked improvement of atopic dermatitis symptoms (dryness, infection, redness) was observed in all subjects after a treatment with TOPIC Medis Extra Moisturizing Gel.

### Example 8

In order to learn about the clinical efficacy of the new formulations over the previous formulations, clinical evaluation of the new and previous formulations of three products from TOPIC Medis line: Face Cream, Body Lotion/cream, Calming Lotion (described above) were examined.

People suffering from atopic dermatitis or sensitive skin were instructed to apply TOPIC Medis Face Cream, TOPIC Medis Body Lotion/Cream or TOPIC Medis Calming Lotion. TOPIC Medis Face Cream or TOPIC Medis Body Lotion/Cream were applied twice a day: Both the previous formulation and the new formulation were applied by the patient at the same time. The previous formulation was applied to the right side of the face or the body and new formulation to the left side of the patient, for a period of minimum 7 days. Effect of the TOPIC Medis Calming Lotion was evaluated when new or previous formulation was applied to a single lesion for one or more times, as needed. Basic demographic data was collected: age, sex, initials.

The patient were later filled an evaluation questionnaire for either the TOPIC Medis Face Cream or the TOPIC Medis Body Lotion/Cream, which included the following questions:

1. Effect of the product on the atopic dermatitis (AD) extent

2. Effect of the product on itch feeling

3. Effect of the product on dryness feeling

4. Effect of the product on skin redness

5. Effect of the product on skin rash

6. Effect of the product on skin appearance

7. General evaluation of the product effect

8. Product texture

9. Product feeling

Questions 1-7 were evaluated as follows: no effect, minimal effect, significant effect. Questions 8, 9 were evaluated as follows: bad, OK, good, excellent

Evaluation questionnaire for TOPIC Medis Calming Lotion included the following questions:

1. Effect of the product on itch feeling

2. Effect of the product on dryness feeling

3. Effect of the product on skin redness

4. General evaluation of the product effect

5. Product texture

6. Product feeling

Questions 1-4 were evaluated as follows: no effect, minimal effect, significant effect. Questions 5, 6 were evaluated as follows: bad, OK, good, excellent

### Results

### TOPIC Medis Face Cream

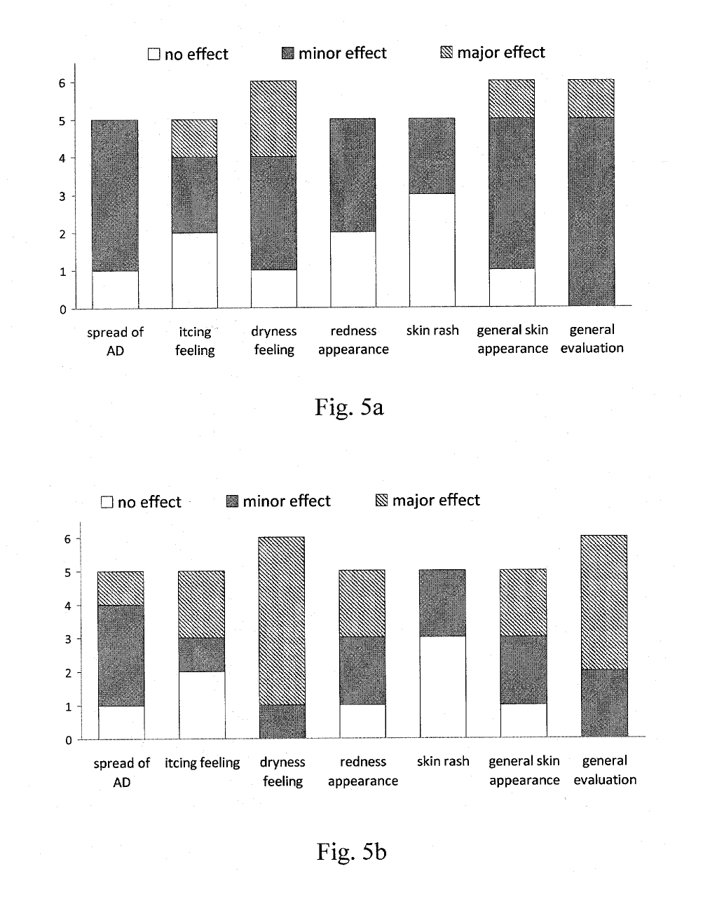

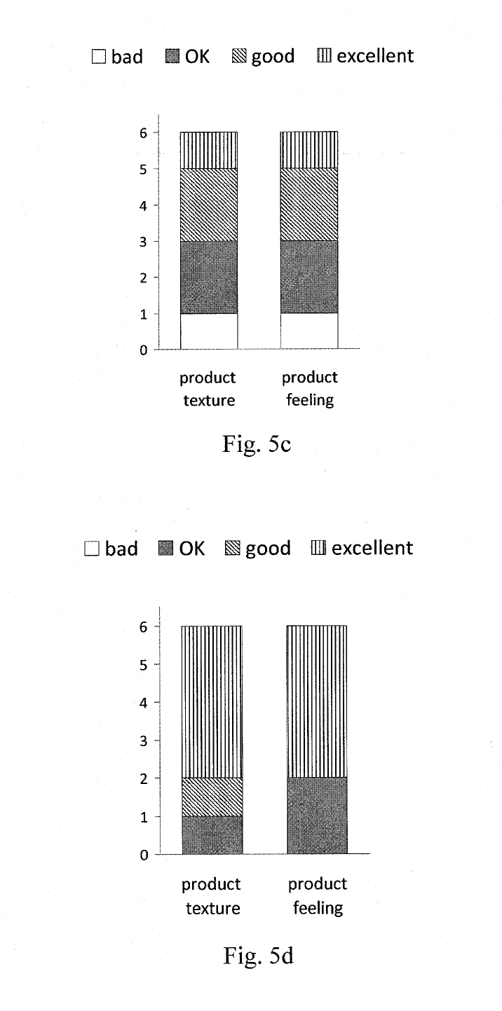

FIGS. 5a, 5b, 5c and 5d show the results of six women at ages 50±13, who applied the previous formulation cream to the right side of the face and the new formulation to the left side of the face, twice a day for 7 to 14 days. FIG. 5a represents the responses to the evaluation questionnaire (questions 1 to 7) after the previous formulation cream was applied on the right side of the face. FIG. 5b represents the responses for the evaluation questionnaire (questions 1 to 7) for the new formulation cream applied to the left side of the face. FIG. 5c represents the responses to questions 8 and 9 of the evaluation questionnaire for the previous formulation cream applied on the right side of the face and FIG. 5d represents the responses for questions 8 to 9 for the new formulation cream applied on the left side of the face.

As can be seen in FIGS. 5A-D the new formulation cream of the TOPIC Medis Face Cream was found more effective that the previous formulation in all parameters except skin rash.

### TOPIC Medis Body Lotion/Cream

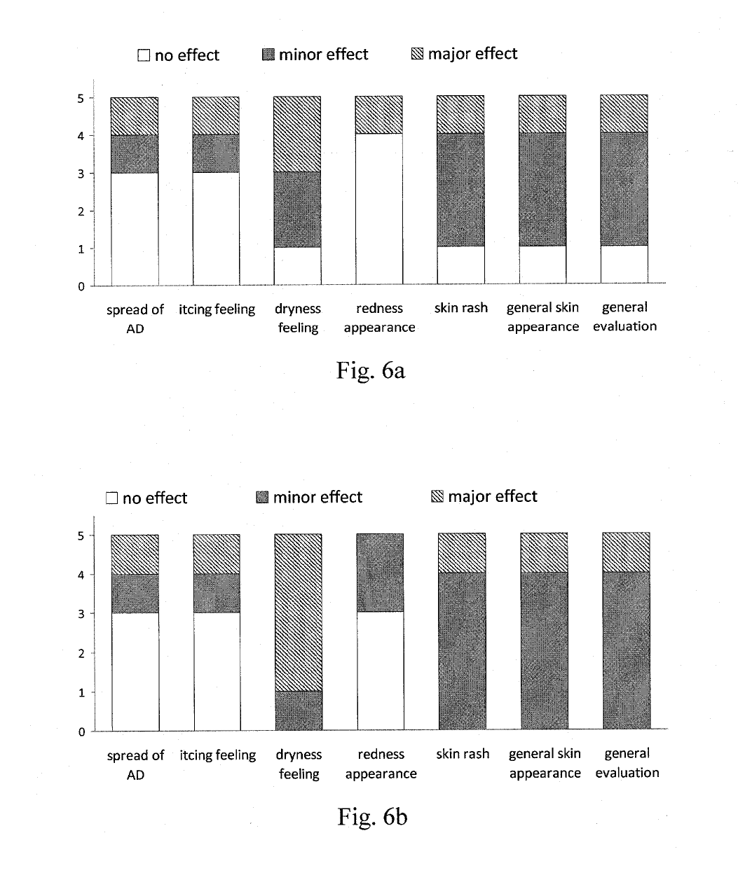

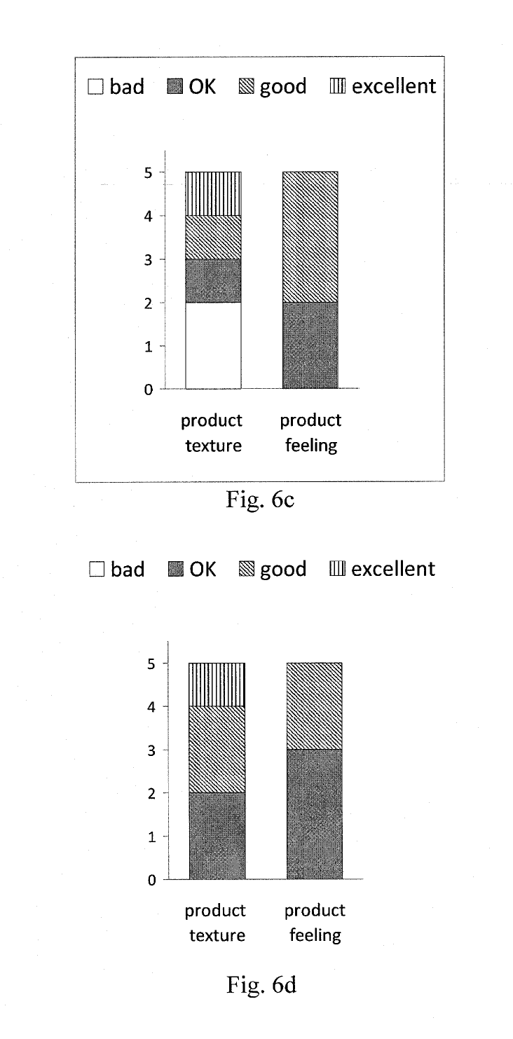

FIGS. 6a, 6b, 6c and 6d show the results of four women and one man at average ages of 37, who applied the previous formulation Lotion to the right side of the body and the new formulation applied to the left side of the body, twice a day for 7 to 14 days. FIG. 6a represents the responses to questions 1-7 of the evaluation questionnaire for the previous formulation Lotion applied on the right side of the body, whereas FIG. 6b represents the responses for the new formulation lotion applied to the left side of the body. FIG. 6c represents the responses to questions 8 and 9 of the evaluation questionnaire (questions 8 to 9) whereas FIG. 6d represents the responses after the new formulation lotion was applied on the left side of the body.

As can be seen in FIG. 6 A-D, the new formulation lotion of the TOPIC Medis Body Lotion was found more effective in all parameters except from AD spread itching feeling.

### TOPIC Medis Calming Lotion

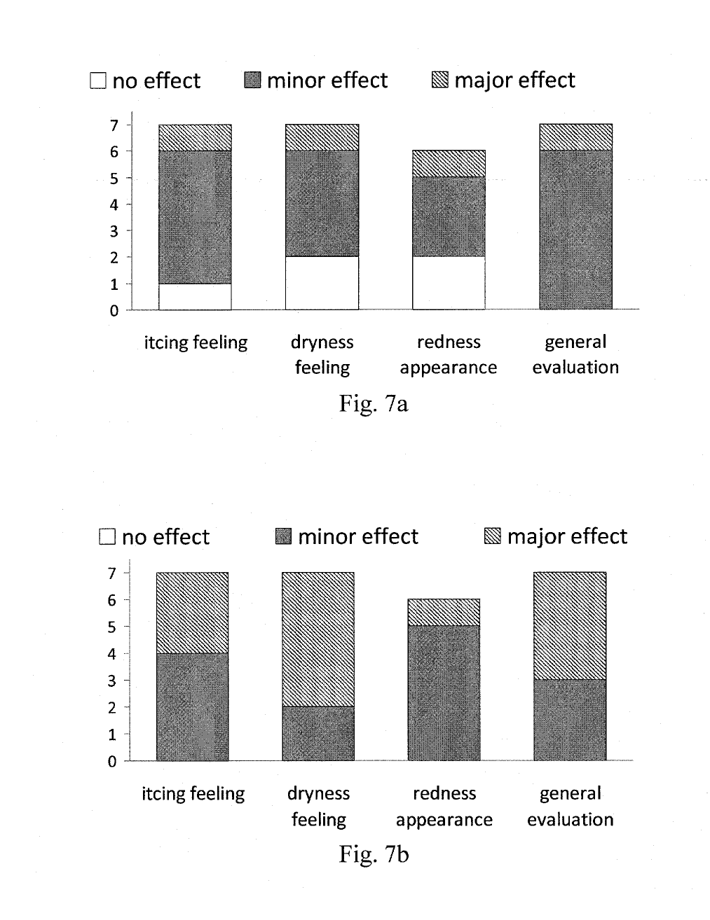

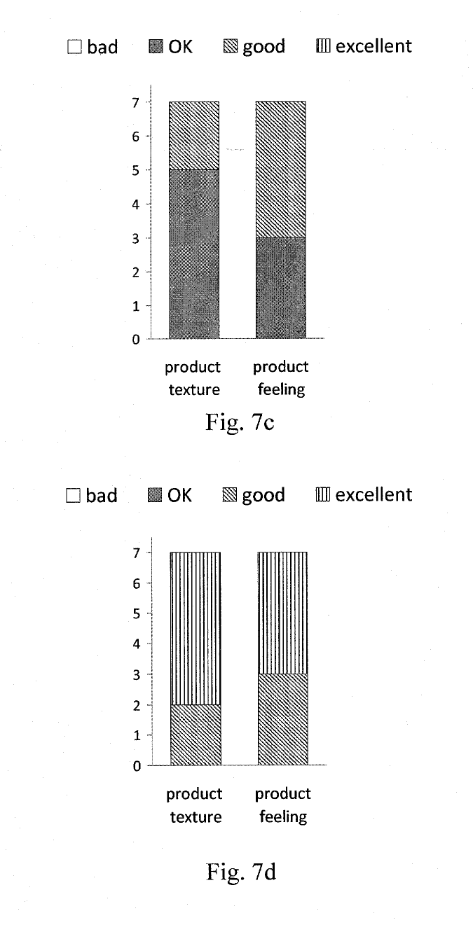

FIGS. 7a, 7b, 7c and 7d show the results of seven women at average ages 38.5. Effect of the TOPIC Medis Calming Lotion was evaluated when new or old formulation was applied on a single lesion for one or more times, as needed. FIG. 7a represents the responses to questions 1 to 4 of the evaluation questionnaire for the previous formulation Calming Lotion that was applied to the right side of the body. FIG. 7b represents the responses to the same questions regarding the new formulation of Calming Lotion which was applied on the left side of the body. FIG. 7c represents the responses to questions 5 and 6 of the evaluation questionnaire regarding the previous formulation Calming Lotion which was applied on the right side of the body. FIG. 7d represents the responses to the same questions for the new formulation Calming Lotion applied which was applied to the left side of the body.

FIGS. 7a, 7b, 7c and 7d show that the new formulation lotion was found more effective in all parameters than the previous formulation.

The results presented in this example clearly shows that the modification of the formulations contributed to higher efficacy of the product. The formulations that comprise the herbal mixture together with additional active ingredients such as hyaluronic acid, a mixture of cholesterol, ceramides and phytosphingosine, moisturizers (avocado oil, shea butter) hydroxyphenyl propamidobenzoic acid and bisabolol improve the efficacy of the formulations and can therefore can act synergistically for atopic dermatitis/sensitive skin treatment.

Moreover, according to the results of the questionnaire the new formulations were evaluated as having a better texture and feeling in comparison to the previous formulations.

1-27. (canceled)

28. A topical composition comprising Rheum palmatum Root Extract, Cnidium monnieri Fruit Extract, Scutellaria baicalensis Root Extract, dipotassium glycyrrhizate, hyaluronic acid and a dermatologically acceptable carrier.

29. The composition according to claim 28, wherein the concentration of the hyaluronic acid ranges from 0.001 to 0.2% w/w.

30. The composition according to claim 29, wherein the concentration of the hyaluronic acid ranges from 0.01 to 0.03% w/w.

31. The composition of claim 28, wherein the composition further comprises cholesterol, ceramide mixture or phytosphingosine or any combination thereof.

32. The composition of claim 28, wherein the composition further comprises emollients.

33. The composition of claim 28, wherein the composition further comprises one or more of sodium lauroyl lactylate, carbomer, xanthan gum or any combination thereof.

34. The composition of claim 28, wherein the composition further comprises tocopheryl acetate.

35. The composition of claim 28, wherein the composition further comprises collodial oatmeal.

36. The composition of claim 28, wherein the composition further comprises glycerine, shea butter (Butyrospermum Parkii) or a combination thereof.

37. The composition of claim 28, wherein the composition further comprises hydroxyphenyl propamidobenzoic acid.

38. The composition claim 28, wherein the composition further comprises bisabolol.

39. The composition according to claim 28, wherein the composition further comprises a skin conditioning agent.

40. The composition according to claim 39, wherein the skin conditioning agent is selected from the group consisting of petrolatum, caprylic/capric triglyceride, cetearyl ethylhexanoate, dimethicone, butyrospermum parkii (shea butter), mineral oil, glycerin, persea gratissima (avocado) oil and phenyl trimethicone or any combination thereof.

41. The composition according claim 28, wherein the composition further comprises emulsifier and/or a viscosity increasing agent.

42. The composition according to claim 41, wherein the emulsifier and/or a viscosity increasing agent is selected from the group consisting of cetearyl alcohol, cetyl alcohol, cetearyl glucoside, glyceryl stearate, PEG-100 stearate, ceteareth-33, sodium acrylate/sodium acryloyldimethyl taurate copolymer, polysorbate 80, polyacrylate 13 & polyisobutene & polysorbate 20 and acrylates/C10-30 alkyl acrylate crosspolymer or any combination thereof.

43. The composition of claim 28, wherein the composition is in a form of oil-in-water, water-in-oil, water-in-oil-in-water, and oil-in-water-in-silicone emulsions, a cream, an ointment, an aqueous solution, a lotion, a soap, a paste, a foam, an emulsion, a gel, a salves, an oil, a wash, a shampoos, a conditioners or an aerosol.

44. A method of treating and/or preventing a chronic inflammatory skin disease or disorder or allergic skin conditions comprising the step of contacting the skin of a subject in need with the composition of claim 28.

45. The method of claim 44, wherein the chronic inflammatory skin disease or disorder is dermatitis.

46. The method of claim 45, wherein the dermatitis is atopic dermatitis, senile dermatitis, radiation dermatitis or contact dermatitis, urticaria or sensitive skin.

47. A method of treating and/or preventing a secondary infection resulted from atopic or contact dermatitis comprising the step of contacting the skin of a subject in need with the composition of claim 28.
